(19) United States
(12) Patent Application Publication
(10) Pub. No.: US 2005/0109345 A1
(43) Pub. Date: May 26, 2005

(54) Laryngeal mask assemblies

(76) Inventors: Michael Norman Collins, Folkestone (GB); Eric Pagan, Hythe (GB)

Correspondence Address: LOUIS WOO;LAW OFFICE OF LOUIS WOO, 717 NORTH FAYETTE STREET, ALEXANDRIA, VA, 22314, US

(21) Appl. No.: 10/986,143

(22) Filed: Nov. 12, 2004

Related U.S. Application Data
(63) Continuation of application No. 09/989,393, filed on Nov. 21, 2001.

(30) Foreign Application Priority Data
Dec. 22, 2000 (GB) 0031661.2

(51) Int. Cl. A61M 16/00

(52) U.S. Cl. 128/207.15; 128/200.26

(57) ABSTRACT

A laryngeal mask has a tube and mount integrally molded from polyurethane as a single piece. A sealing cuff is attached to the mount separately. An inflation line extends in a groove along the outside of the tube and opens at one end into the cuff so that it can be inflated and deflated.

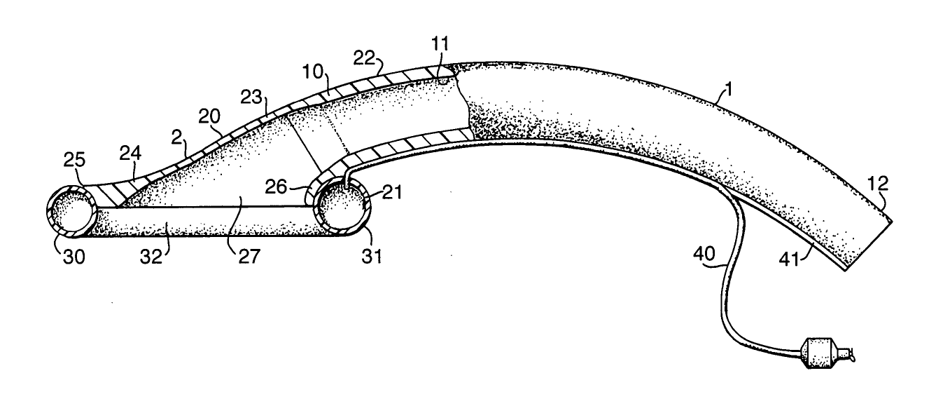

### BACKGROUND OF THE INVENTION

This invention relates to laryngeal mask assemblies and their manufacture.

It is common practice to use an airway known as a laryngeal mask for administering anaesthetic and ventilation gases to a patient. These airways comprise a tube with an inflatable mask or cuff at one end, the tube being inserted in the patient's mouth so that one end is located in the hypopharynx and so that the mask forms a seal in this region with the surrounding tissue. Laryngeal masks are described in, for example, U.S. Pat. No. 5,355,879, U.S. Pat. No. 5,305,743, U.S. Pat. No. 5,297,547, U.S. Pat. No. 5,282,464, GB 2267034, U.S. Pat. No. 5,249,571, U.S. Pat. No. 5,241,956, U.S. Pat. No. 5,303,697, GB 2249959, GB 2111394, EP 448878, U.S. Pat. No. 4,995,388, GB 2205499, GB 2128561, GB 2298797, GB 2334215, GB2337020, PCT/GB/03044, PCT/GB00/03045, GB 0002805 and GB 0020274. Laryngeal masks usually comprise a curved, extruded tube, a separate mount member joined at the patient end of the tube and an inflatable cuff attached to the mount member.

Laryngeal masks have several advantages over endotracheal tubes, which are longer and seal with the trachea below the vocal folds. The multiple components and assembly operations needed to make the masks, however, add to their cost.

It is an object of the present invention to provide an alternative laryngeal mask assembly.

According to one aspect of the present invention there is provided a laryngeal mask assembly comprising a tube, a mount at the patient end of the tube, and an annular sealing cuff extending around the patient end of the mount, the tube and mount being molded together as an integral, single-piece component.

The sealing cuff may be attached with the mount by an adhesive. The sealing cuff is preferably inflatable and deflatable by means of an inflation line, the inflation line extending in a groove along the outside of the tube. The tube and mount may be molded of polyurethane.

According to another aspect of the present invention there is provided a method of manufacture of a laryngeal mask assembly comprising the steps of molding a tube and a mount integrally with the tube and subsequently attaching a sealing cuff with the mount.

According to a further aspect of the present invention there is provided a laryngeal mask assembly made by the method of the above other aspect of the invention.

A laryngeal mask assembly according to the present invention will now be described, by way of example, with reference to the accompanying drawing.

### BRIEF DESCRIPTION OF THE DRAWING

The drawing is a side elevation view of the assembly.

### DETAILED DESCRIPTION OF THE PREFERRED EMBODIMENT

The laryngeal mask assembly comprises a tube 1 and a mask formation 2 at the patient end 10 of the tube.

The tube 1 is of a bendable plastics material, such as PVC and is curved along its length. A bore 11 extends along the tube from its patient end 10 to its rear, machine end 12.

The mask 2 comprises a mount 20 and an inflatable sealing cuff 21. The mount 20 is of a relatively stiff plastics material and is of generally shoe shape. The mount 20 and tube 1 are molded together, such as by injection molding, to form an integral, single piece 22. The mount 20 tapers outwardly from its machine end 23 to its patient end 24, which is inclined to the axis of the machine end at an angle of about 25° so that the patient end of the mount has an oval shape with its forward end 25 being more pointed than its rear end 26. The patient end 24 of the mount 20 is inclined to face towards the inner side of the curve of the tube 1. Internally, the mount 20 has a cavity 27 that increases in cross-sectional area along its length, from the machine end.

The cuff 21 is tubular and of a thin flexible plastics material. The cuff 21 is formed into an annulus of the same shape as the patient end 24 of the mount 20 so that it is oval with its forwardly-directed end 30 being more pointed than its rearwardly-directed end 31. The cuff 21 encloses a central region 32 of the same shape as the patient end 24 of the mount 20. The cuff 21 is attached around the patient end 24 of the mount 20 such as by means of an adhesive. The cuff 21 is inflated and deflated by means of an inflation line 40 which is provided by a separate small-bore tube communicating with the interior of the cuff and extending rearwardly along a groove 41 in the outside of the tube. When inflated in position in a patient, the cuff 21 expands to contact patient tissue in the region of the hypopharnyx.

Because the mount and tube are formed in one operation, there are fewer separate components and fewer steps needed to manufacture the assembly. By avoiding the need to bond the mount to the tube, there is no risk of a faulty bond and there is no need to inspect a bond. It is possible to achieve a smooth external profile in the region between the tube and mount, without a step or bump, because there is no connection in this region. Molding the tube and mount together enables the wall thickness or shape to be varied, if desired, at different points along the length of the tube. It is also possible, by molding, to use different materials, such as polyurethane, which present problems with extrusion.

1-7. (canceled)

8. A laryngeal mask assembly comprising: a tube; a mount at a patient end of said tube; and an annular sealing cuff extending around a patient end of said mount, wherein said tube and said mount are molded together by injection molding from a plastics material as an integral, single-piece component, wherein a groove extends along an outside of said assembly opening along its length on an external surface, wherein an inflation line extends in said groove along the assembly, one end of said inflation line extending out of said groove towards a patient end of said groove and opening into said sealing cuff, and wherein said sealing cuff is attached with said mount as a separate component and is adapted to seal with tissue in the region of the hypopharynx.

9. A laryngeal mask assembly according to claim 8, wherein said sealing cuff is attached with said mount by an adhesive.

10. A laryngeal mask assembly according to claim 8, wherein said airway tube and mount are molded of polyurethane.

11. A method of forming a laryngeal mask assembly comprising the steps of:
injection molding a tube and a mount at a patient end of said tube together from a plastics material as an integral, single-piece component; and
extending an annular sealing cuff around a patient end of said mount.

12. The method of claim 11, further comprising the steps of:
extending a groove along an outside of said tube opening along its length on an external surface;
extending an inflation line in said groove along the assembly, one end of said inflation line extending out of said groove towards a patient end of said groove and opening into said sealing cuff.

13. The method of claim 11, further comprising the steps of:
attaching said sealing cuff with said mount as a separate component; and
adapting said sealing cuff to seal with tissue in the region of the hypopharynx.

14. A laryngeal mask assembly comprising: a tube; a mount at a patient end of said tube, said mount having a shaped patient end; and an annular sealing cuff formed into an annulus having the same shape as the patient end of said mount attaching around the patient end of said mount, said tube and said mount being molded together from a plastics material as an integral, single-piece component, wherein an inflation line extends along the assembly, one end of said inflation line extending towards a patient end and opening into said sealing cuff, and wherein said sealing cuff and is adapted to seal with tissue in the region of the hypopharynx.

15. Laryngeal mask assembly according to claim 14, wherein said sealing cuff attaches around the patient end of said mount by circumferentially covering the edge at the patient end of said mount.

16. Laryngeal mask assembly according to claim 14, wherein said tube and said mount are integrally formed as one component by injection molding.

17. Laryngeal mask assembly according to claim 14, wherein said cuff is attached to said mount as a separate component.

18. Laryngeal mask assembly according to claim 14, wherein said cuff is attached to said mount by an adhesive.

19. A laryngeal mask assembly comprising an integral one piece tube having a patient end that tapers to form a mount, and an annular sealing cuff extending around said mount, said cuff being formed of a thin flexible plastics material so as to be inflatable and is adapted to seal with tissue in the region of the hypopharynx.

20. A laryngeal mask assembly according to claim 19, wherein said mount has a cavity that decreases in cross sectional area from said patient end.

21. A laryngeal mask assembly according to claim 19, wherein said sealing cuff is attached with said mount by an adhesive.

22. A laryngeal mask assembly according to claim 19, wherein said tube further has a machine end, said tube having a thickness that is substantially uniform from said machine end to the portion of said tube that forms said mount, the thickness of the tapered portion of said mount being thinner than the portion of said tube that has the substantially uniform thickness.

23. A laryngeal mask assembly according to claim 19, wherein a groove extends along an outside of said tube opening along its length on an external surface, wherein an inflation line extends in said groove along said tube.
